(19) United States
(12) Patent Application Publication
(10) Pub. No.: US 2009/0088732 A1
(43) Pub. Date: Apr. 2, 2009

(54) Rapid Refill Programming for Implantable Drug Pump

(76) Inventor: Daniel Hernandez Villegas, Grenada Hills, CA (US)

Correspondence Address: HENRICKS SLAVIN AND HOLMES, LLP;ADVANCED BIONICS CORPORATION, 840 APOLLO STREET, SUITE 200, EL SEGUNDO, CA, 90245, US

(21) Appl. No.: 11/866,300

(22) Filed: Oct. 2, 2007

(51) Int. Cl. A61K 9/22

(52) U.S. Cl. 604/891.1

(57) ABSTRACT

A programming device for an implantable drug pump includes a display device, a communication device, and a controller. The communication device is adapted to facilitate a communication link between the programming device and an implantable drug pump. The controller is adapted to receive identification information for the implantable drug pump when the communications link has been established and, when the implantable drug pump has been identified, to control the display device to generate a graphical user interface which permits a user of the programming device to initiate a refill procedure for the implantable drug pump.

### BACKGROUND OF THE INVENTION

1. Field of Invention

The present invention relates generally to implantable medical devices and, in particular, to refilling an implantable drug pump with medicine.

2. Description of the Related Art

Implantable drug delivery systems are subject to regular therapy management and pump maintenance. A significant and regular event in pump maintenance is the refilling of the pump with medication. For example, morphine remains stable in the pump for up to 90 days; therefore, the morphine in the pump must be replaced within 90 days. However, the frequency of refilling of the pump typically varies depending on patient therapy, medication, drug usage, drug concentration, and physician practice.

For known current implantable drug pumps that do not have a reservoir level sensor to measure the fluid level in their reservoir, delivered medication amount can be tracked by a controller to provide an indication of reservoir level. In such systems, clinicians are required to use a programming device to communicate to the pump that the pump has been refilled and to what level. Therefore, a complete refilling procedure requires the physical activity of refilling the pump, as well as programming of the pump. Unfortunately, known programming devices are often complex and/or require an excessive amount of human-to-programming device interaction.

It would be helpful to be able to provide a programming device that provides a simple, user-friendly interface for programming the refilling of a pump. It would also be helpful to be able to provide a programming device that reduces the amount of human-to-programming device interaction. It would also be helpful to be able to provide a programming device that addresses the foregoing while retaining the flexibility of more advanced programming features.

### SUMMARY OF THE INVENTION

In an example embodiment, a programming device for an implantable drug pump includes a display device, a communication device, and a controller. The communication device is adapted to facilitate a communication link between the programming device and an implantable drug pump. The controller is adapted to receive identification information for the implantable drug pump when the communications link has been established and, when the implantable drug pump has been identified, to control the display device to generate a graphical user interface prompting a user of the programming device to provide a single user input that initiates a standard refill procedure for the implantable drug pump.

In an example embodiment, a programming device for an implantable drug pump includes a display device, a communication device, and a controller. The communication device is adapted to facilitate a communication link between the programming device and an implantable drug pump. The controller is adapted to receive identification information for the implantable drug pump when the communications link has been established and, when the implantable drug pump has been identified, to control the display device to generate a graphical user interface which permits a user of the programming device to initiate a refill procedure for the implantable drug pump.

The above described and many other features of the present invention will become apparent as the inventions become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 illustrates an implantable medical device and example programmers/controllers, which embody the implantable drug pump programming devices described herein.

FIG. 2 is a plan view of a programming device in accordance with one embodiment of the present invention, shown establishing a communications link with an implantable medical device.

FIG. 3 is a block diagram of the programming device and implantable medical device of FIG. 2.

FIG. 4 is a plan view of a programming device in accordance with another embodiment of the present invention.

FIG. 5 is a plan view of a programming device in accordance with still another embodiment of the present invention.

FIGS. 6A and 6B are a flow chart in accordance with one embodiment of the present invention.

FIG. 7 shows a graphical user interface (including dosage and refill user interfaces) generated by a programming device according to an example embodiment of the present invention.

FIG. 8 shows a graphical user interface (including a daily profile adjustments user interface and a graphical representation of a daily profile overview) generated by a programming device according to an example embodiment of the present invention.

### DETAILED DESCRIPTION OF THE EXEMPLARY EMBODIMENTS

The following is a detailed description of the best presently known modes of carrying out the invention. This description is not to be taken in a limiting sense, but is made merely for the purpose of illustrating the general principles of the invention.

The present remote controls or programmers have application in a wide variety of medical device systems. One example of such a system is an implantable infusion device system and the present invention is discussed in the context of implantable infusion device systems. The present invention is not, however, limited to implantable infusion device systems and is instead also applicable to other medical device systems that currently exist, or are yet to be developed. For example, the present invention is applicable to other ambulatory medical device systems. Such systems include, but are not limited to, externally carried infusion pump systems, implantable pacemaker and/or defibrillator systems, implantable neural stimulator systems, and implantable and/or externally carried physiologic sensor systems.

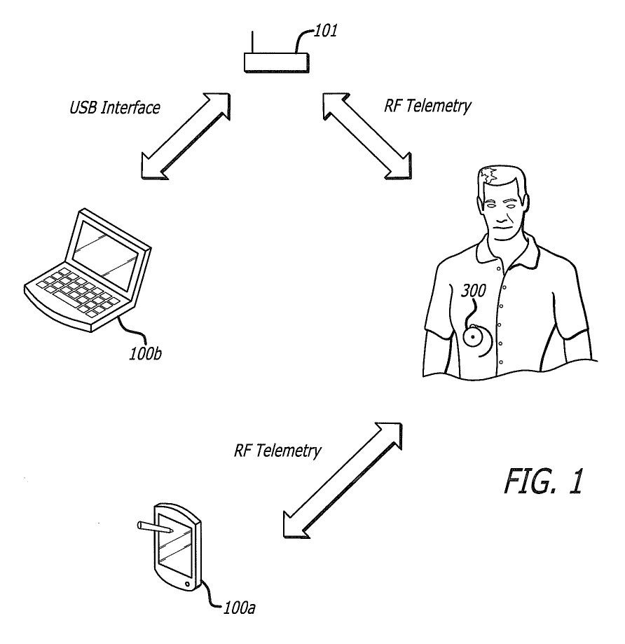

One example of a programmer in accordance with the present invention is an implantable infusion device system. The implantable infusion device system may include any one of the remote controls or programmers described herein in combination with an implantable infusion device. FIG. 1 illustrates an implantable medical device 300 and example programmers/controllers, which embody the drug delivery safety systems described herein. The example programmers/controllers include a programmer 100a (such as a portable computing device (PCD) or personal digital assistant (PDA)) and a clinician programmer 100b (such as a clinician programmer/field support system). In this example, the programmer 100a includes a communication device which facilitates radio frequency (RF) communications with the implantable medical device 300 so that RF telemetry can be communicated between the devices. Also in this example, the clinician programmer 100b is connected to a programmer interface module 101 with a USB Interface; the programmer interface module 101, in turn, facilitates RF communications between the clinician programmer 100b and the implantable medical device 300. It should be understood that other types of programmers/controllers as well as other communications interfaces can also be employed.

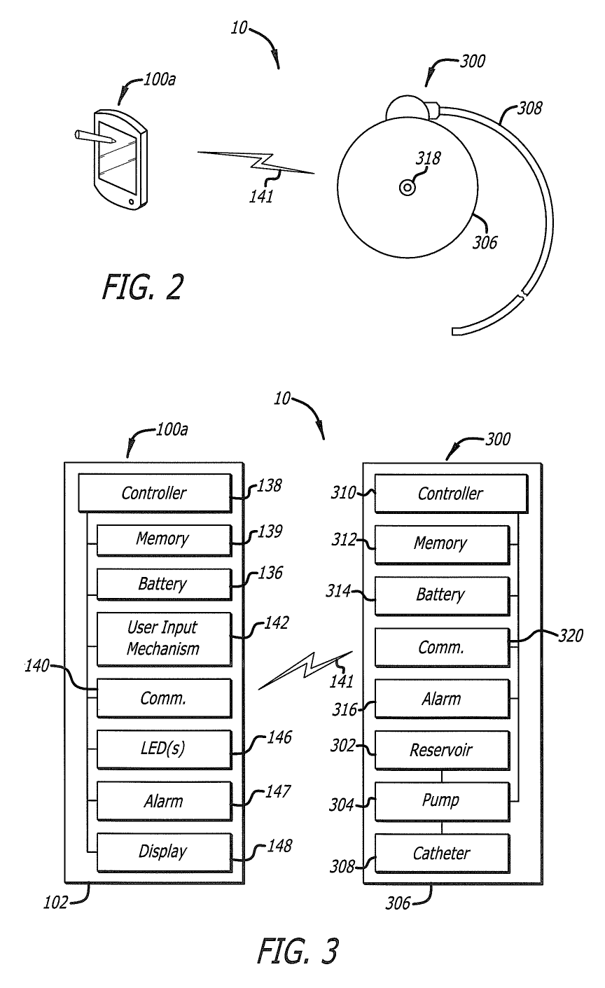

Referring to FIG. 2, in an example embodiment, an implantable medical device system 10 includes a programmer 100a and an implantable medical device 300. In an example embodiment, the programmer 100a includes a battery or other power source 136, a controller 138, such as a microprocessor, microcontroller or other control circuitry, memory 139, a user input mechanism 142 (such as a keyboard, mouse, touch screen and/or voice recognition device), one or more LEDs 146 (and/or alarm 147), and a display 148. The memory 139 can also be contained within the controller 138 (e.g., within a microcontroller). By way of example and not of limitation, the alarm 147 can include one or more of an audio speaker and a vibration device. A communication device 140 (including an antenna if necessary) is also provided. In an example embodiment, the display 148 is a touch screen configured to receive user inputs, i.e., at least a portion of the functionality of the user input mechanism 142 is provided by the display 148.

The communication device 140 establishes a communications link 141 (e.g., an RF communications link) with the implantable medical device 300. Although the present invention is not limited to any particular communication device, in an example embodiment, the communication device 140 is a telemetry device that transmits an RF signal at a specified frequency or set of frequencies. The RF signal may, in some instances, be a carrier signal that carries bit streams. The communication device 140 is also configured to receive signals from the implantable medical device 300. Other exemplary communication devices include oscillating magnetic field communication devices, static magnetic field communication devices, optical communication devices, ultrasound communication devices and direct electrical communication devices.

In this example embodiment, the implantable medical device 300 is an implantable infusion device and includes a medication reservoir 302 and a pump or other fluid transfer device 304 within a housing 306. The pump 304 transfers medication from the reservoir 302 through a catheter 308 to the target region within the body. Operation of the implantable medical device 300 is controlled by a controller 310, such as a microprocessor, microcontroller or other control circuitry, in accordance with instructions stored in memory 312. Power is provided by a battery or other power source 314. An alarm 316 (e.g., an audible alarm such as an audio speaker, and/or a vibration device) may also be provided in order to inform the patient, for example, when the amount of medication in the reservoir 302 is low or when the amount of energy stored in the battery 314 is low. A refill port 318, which allows the reservoir to be refilled while the implantable medical device 300 is within the patient, is positioned on the exterior of the housing 306.

A communication device 320 is also provided. In this example embodiment, the communication device 320 is configured to receive signals from, and transmit signals to, the programmer 100a. In an example embodiment, the communication device 320 is a telemetry device that transmits and receives RF signals at a specified frequency or set of frequencies. The RF (or other) signal may, in some instances, be a carrier signal that carries bit streams.

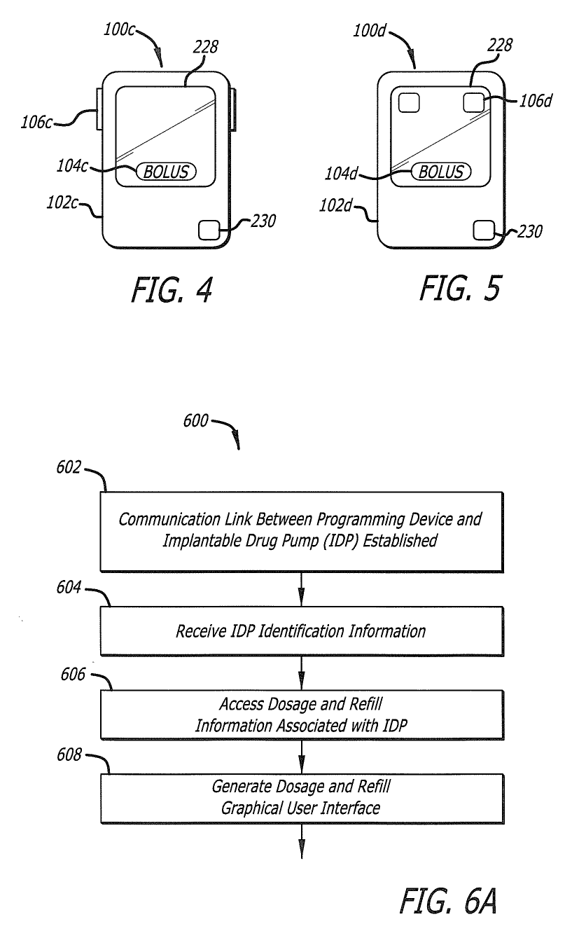

It should be noted here that, in the context of the present invention, different types and/or combinations of user input devices can be employed with any given programmer/controller device. As illustrated for example in FIG. 4, the exemplary programmer 100c includes a housing 102c and a touch screen 228. A controller and a communication device (not shown) are also provided. The touch screen 228 may be used to display one or more button configurations in order to allow the user to accomplish various tasks. At least one of the displayed buttons is a bolus delivery button 104c. The housing 102c may also be provided with one or more button control elements 106c (e.g. buttons), which are operably connected to the controller, and a power on/off button 230.

One or more button control elements may, alternatively, be provided on a touch screen. Turning to FIG. 5, the exemplary programmer 100d includes a housing 102d, a touch screen 228 that may be used to, among other things, display a bolus delivery button 104d and a pair of button control elements 106d, and a power on/off button 230.

Referring again to FIG. 3, in this example embodiment, the controller 138 and memory 139 are contained within the housing 102 of the programmer 100a. The scope of the present invention also includes programmers or programming systems where the functionality of the controller 138, or a portion of this functionality, is “migrated” to a physical location that is external to the housing 102. Similarly, some or all of the memory 139 can be physically located external to the housing 102. Such external controller(s) and memory device(s) can be operatively interfaced with the programmer 100a with wireless or wired communication links.

In an example embodiment, a database of information relating to the implantable medical device 300 (e.g., an implantable drug pump) is stored in the memory 139. In an example embodiment, a baseline database of information is initially uploaded into the memory 139. In an example embodiment, the controller 138 manages the function of uploading a database of information, as well as writing changes, additions or updates to the database of information. Under control of the controller 138 and in response to user inputs provided via the user input mechanism 142, the programmer 100a performs the various functions described herein, in particular, providing an interactive user interface, e.g., a graphical user interface (GUI), at the display 148.

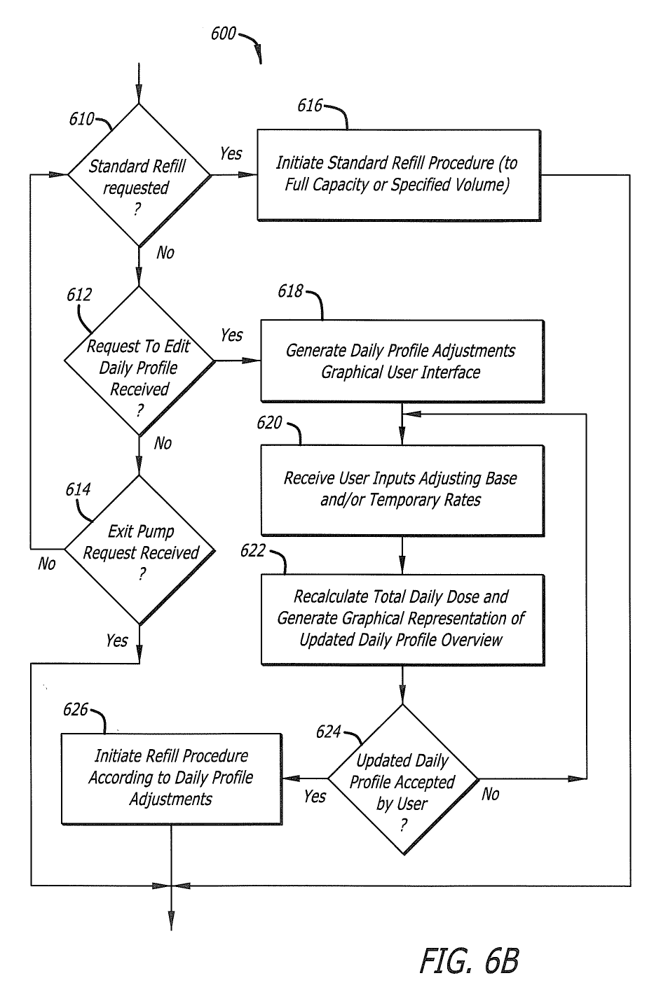

Referring to FIGS. 6A and 6B, in an example embodiment, a method 600 of programming a refill procedure for an implantable drug pump (IDP) is now described. At 602, a communication link between a programming device and an implantable drug pump (IDP) is established. At 604, information uniquely identifying the IDP is received. In an example embodiment, the programming device (e.g., programmer 100a) is adapted to automatically recognize a pump that is within communications range. In another example embodiment, the programming device is not capable of recognizing a pump within communications range, rather the clinician is provided with a user option on the screen to recognize (uniquely identify) the pump.

At 606, dosage and refill information associated with the IDP is accessed. In an example embodiment, the programming device obtains therapeutic data contained within the identified implanted device (e.g., IDP). At 608, in this example embodiment, a dosage and refill graphical user interface is generated and presented to the user on the display 148. In an example embodiment, the programming device automatically generates a dosage and refill graphical user interface (once the pump has been identified). In another example embodiment, a screen with the following information is generated:

1. Patient Name.

2. Pump ID.

3. Medication names and associated concentrations.

4. Reservoir capacity.

5. Daily Delivery Dosages.

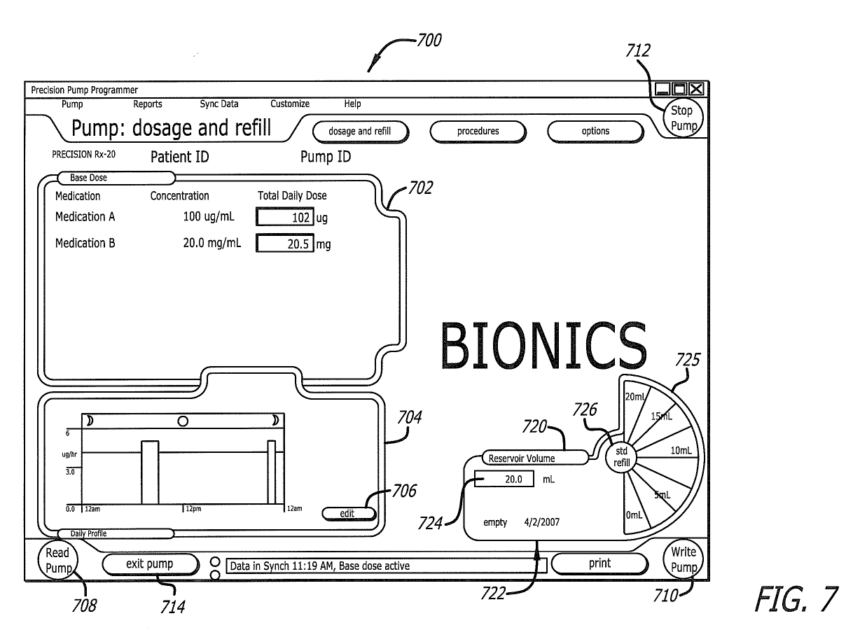

In an example embodiment, at 608, the programming device generates a graphical user interface 700 (FIG. 7) including dosage and refill user interfaces. In this example embodiment, the graphical user interface 700 is generated at the display 148. In this example embodiment, the graphical user interface 700, denoted “Pump: dosage and refill”, displays patient and pump identification information (representatively denoted as “PatientID” and “PumpID”) to assist the user in verifying that communications have been established with the correct pump. In this example embodiment, the graphical user interface 700 includes a Base Dose display area 702, which displays a table of information for Base Dose medications. More specifically, in this example embodiment, Base Dose display area 702 shows a table of information organized under the headings: Medication, Concentration, and Total Daily Dose. In this example embodiment, the graphical user interface 700 also includes a Daily Profile display area 704 in which a daily profile for one or more drugs is shown, with drug(s) concentration plotted over time. This graphical presentation of the daily profile provides the clinician or other user of the programmer 100a with a tool for assessing possible adjustments to the daily profile.

Thus, in an example embodiment, the graphical user interface 700 includes one or more fields in which additional information is displayed. By way of example, the additional information includes medication information for a patient associated with the implantable drug pump. The medication information can include, but is not limited to, base dose information, patient-controlled analgesia (PCA) dose information, and/or a daily medication profile (e.g., information relating to a graphical representation of a daily medication profile).

In an example embodiment, the programmer 100a is configured to generate the graphical user interface 700 such that the Total Daily Dose (for each Medication) shown in the Base Dose display area 702 can be adjusted by entering new dosages. In another example embodiment, the plot generated in the Daily Profile display area 704 is automatically adjusted depending upon the dosages entered into the fields in the Total Daily Dose column. In an example embodiment, an edit button 706 in the Daily Profile display area 704, when actuated, permits a user to edit the daily profile. In another example embodiment, the Total Daily Dose value for each Medication is automatically adjusted depending upon changes made to the daily profile.

In this example embodiment, the graphical user interface 700 also includes a Read Pump button 708, a Write Pump button 710, a Stop Pump button 712, and an Exit Pump button 714, which initiate these respective functions when actuated.

Referring again to FIG. 7, in this example embodiment, the graphical user interface 700 includes a Reservoir Volume display area 720, which displays a calculation of an empty reservoir date in response to a daily dosage change. In this example embodiment, the Reservoir Volume display area 720 includes an empty reservoir date indication field 722 which is updated by the controller 138, e.g., when a daily dosage is changed. In an example embodiment, the empty reservoir date is calculated based on the IDP's current delivery regime and any ongoing delivery procedures. In an example embodiment, the programmer 100a is configured such that the alarm 147 is activated on the empty reservoir date calculated.

The Reservoir Volume display area 720 includes a field 724 which, in an example embodiment, is controlled to display a reservoir volume of medication remaining in the reservoir 302 of the implantable drug pump 300. In this example embodiment, the reservoir volume is also graphically presented by graphical field 725, which is presented as shown as a gage.

In this example embodiment, the Reservoir Volume display area 720 includes a button 726 which can be actuated by a user of the programmer 100a to request a “standard refill” for the implantable drug pump.

In an example embodiment, the term “standard refill” means that the refill procedure is performed to the maximum capacity of the pump. The term “standard refill” can also mean refilling of the pump to a particular percentage of its maximum volume (e.g., 95% full). The term “standard refill” can also mean refilling of the pump with a particular volume of a particular medication. Other definitions of “standard refill” are also within the scope of the present invention.

The graphical user interface 700 includes one or more fields adapted to be actuated to generate user inputs. For instance, in this example embodiment, the button 726, when actuated, caused the controller 138 to initiate a refill procedure (e.g., a standard refill).

In an example embodiment, a programming device for an implantable drug pump includes a display device, a communication device, and a controller. The communication device is adapted to facilitate a communication link between the programming device and an implantable drug pump. The controller is adapted to receive identification information for the implantable drug pump when the communications link has been established and, when the implantable drug pump has been identified, to control the display device to generate a graphical user interface which permits a user of the programming device to initiate a refill procedure for the implantable drug pump.

In an example embodiment, a programming device for an implantable drug pump includes a display device, a communication device, and a controller. The communication device is adapted to facilitate a communication link between the programming device and an implantable drug pump. The controller is adapted to receive identification information for the implantable drug pump when the communications link has been established and, when the implantable drug pump has been identified, to control the display device to generate a graphical user interface prompting a user of the programming device to provide a single user input that initiates a standard refill procedure for the implantable drug pump.

The graphical user interface 700 includes one or more fields adapted to receive data entered by the user. For instance, in an example embodiment, the field 724 is controlled to function as a reservoir volume input field, and the controller 138 is adapted to implement a user specified refill procedure depending upon a value entered by the user in the reservoir volume input field. In this manner, a user can in a simply way essentially override the standard refill protocol to, for example, request a refill volume less than the volume that would be added to the pump during implementation of a standard refill. The graphical user interface 700 is controlled such that a warning or other user prompt is generated should a user attempt to input a refill volume that would result in a volume of medicine being dispensed that exceeds the capacity of the reservoir 302, taking into consideration the amount of medicine already in the reservoir 302.

Referring to FIG. 6B, in this example method 600 of programming a refill procedure for an implantable drug pump (IDP), at 610, a determination is made as to whether a standard refill has been requested (e.g., by actuating the button 726). If standard refill has been requested, at 616, the standard refill procedure is initiated. If standard refill has not been requested, a determination is next made as to whether the user has requested to edit the daily profile (e.g., by actuating the edit button 706). If no request to edit the daily profile has been made, at 614, a determination is then made as to whether the user has requested to exit pump (e.g., by actuating the Exit Pump button 714). However, if a request to edit the daily profile has been made, at 612, the display 148 is controlled to generate a daily profile adjustments user interface.

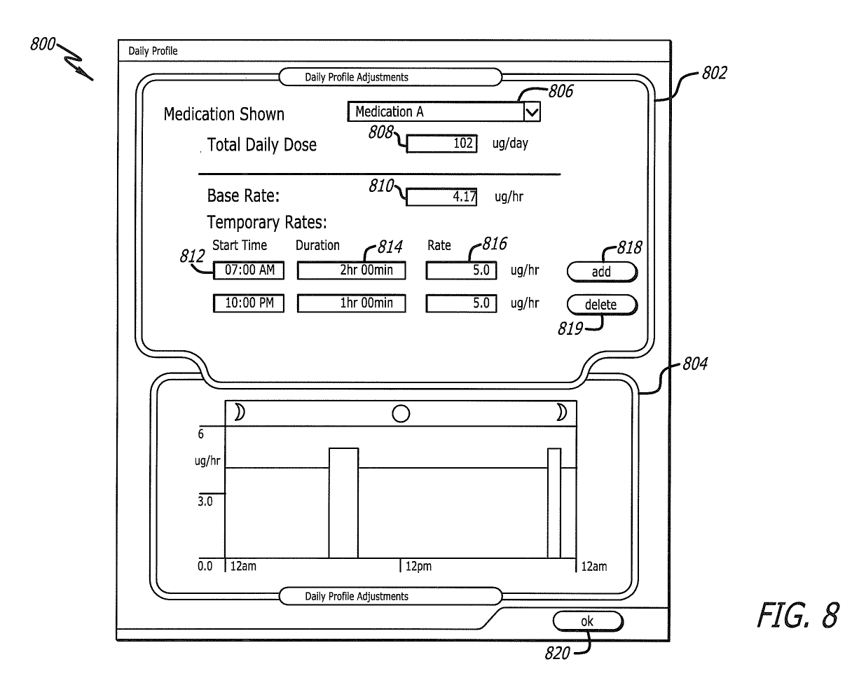

In an example embodiment, at 618, the programming device generates a graphical user interface 800 (FIG. 8) including a daily profile adjustments user interface 802 and a graphical representation of a medication daily profile 804 (denoted “Daily Profile Overview”). In this example embodiment, the graphical user interface 800 is generated at the display 148. In an example embodiment, when the edit button 706 is actuated, this causes the controller 138 to generate the daily profile adjustments user interface 802 which allows the user to make adjustments to a medication daily profile.

In this example embodiment, the daily profile adjustments user interface 802 includes a pull down menu 806 that allows the user to select a medication associated with the medication daily profile, and the controller 138 is adapted to automatically update the daily profile adjustments user interface 802 depending upon the medication selected. In this example embodiment, the daily profile adjustments user interface 802 includes a Total Daily Dose field 808, and the controller 138 is adapted to automatically recalculate and display an updated total daily dosage within the total daily dosage field 808 after an adjustment to the medication daily profile is made.

In this example embodiment, the medication daily profile 804 shows the rate at which medication is dispensed over the course of a day. In an example embodiment, the medication daily profile 804 is defined by a base rate, and can also include one or more intervals during which medication is dispensed at temporary rates. In this example embodiment, the daily profile adjustments user interface 802 includes a Base Rate field 810, Temporary Rate Start Time fields 812, Temporary Rate Duration fields 814, and Temporary Rate fields 816, which allow the user to make adjustments to a base rate and/or temporary rate for a medication associated with the medication daily profile. In an example embodiment, the daily profile adjustments user interface 802 allows the user to specify and add or remove multiple temporary rate intervals to the medication daily profile. In this example embodiment, once a user has entered Start Time, Duration, and Rate values, the add button 818 is actuated to add the newly specified temporary rate interval. In this example embodiment, the controller 138 adjusts the graphical representation of the medication daily profile 804 in response to the adjustments. In an example embodiment, the daily profile adjustments user interface 802 allows the user to specify and remove pre-existing temporary rate intervals to the medication daily profile. In this example embodiment, once a user has selected Start Time, Duration, and Rate values, the delete button 819 is actuated to remove the specified temporary rate interval. In this example embodiment, the controller 138 adjusts the graphical representation of the medication daily profile 804 in response to the adjustments.

Referring again to FIG. 6B, in this example method 600 of programming a refill procedure for an implantable drug pump (IDP), at 620, user inputs adjusting the base and/or temporary rates are received (e.g., at the daily profile adjustments user interface 802). At 622, the controller 138 recalculates the total daily dose and updates the Total Daily Dose field 808 with this value, and generates an updated graphical representation of the medication daily profile 804. At 624, a determination is made as to whether the user has accepted the updated daily profile. If the updated daily profile is accepted, at 626, the controller 138 initiates the refill procedure according to the accepted daily profile adjustments. In this example embodiment, the OK button 820 is actuated to accept the updated daily profile. If the adjustments are not accepted, at 620, the user is given an opportunity to make further adjustments as described above.

Although the invention disclosed herein has been described in terms of the preferred embodiments above, numerous modifications and/or additions to the above-described preferred embodiments would be readily apparent to one skilled in the art. The invention also includes any combination of the elements from the various species and embodiments disclosed in the specification that are not already described. It is intended that the scope of the present invention extend to all such modifications and/or additions and that the scope of the present invention is limited solely by the claims set forth below.

What is claimed is:

1. A programming device for an implantable drug pump, the programming device comprising:
a display device;
a communication device adapted to facilitate a communication link between the programming device and an implantable drug pump; and
a controller adapted to receive identification information for the implantable drug pump when the communications link has been established and, when the implantable drug pump has been identified, to control the display device to generate a graphical user interface prompting a user of the programming device to provide a single user input that initiates a standard refill procedure for the implantable drug pump.

2. The programming device for an implantable drug pump of claim 1, wherein the controller is adapted to automatically attempt to identify the implantable drug pump when the communications link has been established.

3. The programming device for an implantable drug pump of claim 1, wherein the graphical user interface includes one or more fields in which additional information is displayed.

4. The programming device for an implantable drug pump of claim 3, wherein the additional information includes medication information for a patient associated with the implantable drug pump.

5. The programming device for an implantable drug pump of claim 4, wherein the medication information includes base dose information.

6. The programming device for an implantable drug pump of claim 4, wherein the medication information includes patient-controlled analgesia (PCA) dose information.

7. The programming device for an implantable drug pump of claim 4, wherein the medication information includes a daily medication profile.

8. The programming device for an implantable drug pump of claim 7, wherein the daily medication profile is graphically presented.

9. The programming device for an implantable drug pump of claim 3, wherein the additional information includes a reservoir volume of medication remaining in a reservoir of the implantable drug pump.

10. The programming device for an implantable drug pump of claim 9, wherein the reservoir volume is graphically presented.

11. The programming device for an implantable drug pump of claim 1, wherein the graphical user interface includes one or more fields adapted to receive data entered by the user.

12. The programming device for an implantable drug pump of claim 11, wherein the one or more fields include a reservoir volume input field, and the controller is adapted to implement a user specified refill depending upon a value entered by the user in the reservoir volume input field.

13. The programming device for an implantable drug pump of claim 1, wherein the graphical user interface includes one or more fields adapted to be actuated to generate user inputs.

14. The programming device for an implantable drug pump of claim 13, wherein the one or more fields include a standard refill button.

15. The programming device for an implantable drug pump of claim 13, wherein the one or more fields include an edit button which, when actuated, causes the controller to generate a daily profile adjustments user interface that allows the user to make adjustments to a medication daily profile.

16. The programming device for an implantable drug pump of claim 15, wherein the daily profile adjustments user interface includes a pull down menu that allows the user to select a medication associated with the medication daily profile, and the controller is adapted to automatically update the daily profile adjustments user interface depending upon the medication selected.

17. The programming device for an implantable drug pump of claim 15, wherein the daily profile adjustments user interface includes a total daily dosage field, and the controller is adapted to automatically recalculate and display an updated total daily dosage within the total daily dosage field after an adjustment to the medication daily profile is made.

18. The programming device for an implantable drug pump of claim 15, wherein the daily profile adjustments user interface allows the user to make adjustments to a base rate and/or temporary rate for a medication associated with the medication daily profile.

19. The programming device for an implantable drug pump of claim 15, wherein the controller adjusts a graphical representation of the medication daily profile in response to the adjustments.

20. The programming device for an implantable drug pump of claim 15, wherein the daily profile adjustments user interface allows the user to specify and add or remove multiple temporary rate intervals to the medication daily profile.

21. A programming device for an implantable drug pump, the programming device comprising:
a display device;
a communication device adapted to facilitate a communication link between the programming device and an implantable drug pump; and
a controller adapted to receive identification information for the implantable drug pump when the communications link has been established and, when the implantable drug pump has been identified, to control the display device to generate a graphical user interface which permits a user of the programming device to initiate a refill procedure for the implantable drug pump.

22. The programming device for an implantable drug pump of claim 21, wherein the refill procedure is a standard refill procedure for the implantable drug pump.

23. The programming device for an implantable drug pump of claim 22, wherein the graphical user interface includes a standard refill button, and the controller is adapted to initiate the standard refill procedure after the user actuates the standard refill button.

24. The programming device for an implantable drug pump of claim 21, wherein the controller is adapted to automatically attempt to identify the implantable drug pump when the communications link has been established.

25. The programming device for an implantable drug pump of claim 21, wherein the graphical user interface includes one or more fields in which additional information is displayed.

26. The programming device for an implantable drug pump of claim 25, wherein the additional information includes medication information for a patient associated with the implantable drug pump.

27. The programming device for an implantable drug pump of claim 26, wherein the medication information includes base dose information.

28. The programming device for an implantable drug pump of claim 26, wherein the medication information includes patient-controlled analgesia (PCA) dose information.

29. The programming device for an implantable drug pump of claim 26, wherein the medication information includes a daily medication profile.

30. The programming device for an implantable drug pump of claim 29, wherein the daily medication profile is graphically presented.

31. The programming device for an implantable drug pump of claim 25, wherein the additional information includes a reservoir volume of medication remaining in a reservoir of the implantable drug pump.

32. The programming device for an implantable drug pump of claim 31, wherein the reservoir volume is graphically presented.

33. The programming device for an implantable drug pump of claim 21, wherein the graphical user interface includes one or more fields adapted to receive data entered by the user.

34. The programming device for an implantable drug pump of claim 33, wherein the one or more fields include a reservoir volume input field, and the controller is adapted to implement a user specified refill depending upon a value entered by the user in the reservoir volume input field.

35. The programming device for an implantable drug pump of claim 21, wherein the graphical user interface includes one or more fields adapted to be actuated to generate user inputs.

36. The programming device for an implantable drug pump of claim 35, wherein the one or more fields include a refill button which, when actuated, caused the controller to initiate the refill procedure.

37. The programming device for an implantable drug pump of claim 35, wherein the one or more fields include an edit button which, when actuated, causes the controller to generate a daily profile adjustments user interface that allows the user to make adjustments to a medication daily profile.

38. The programming device for an implantable drug pump of claim 37, wherein the daily profile adjustments user interface includes a pull down menu that allows the user to select a medication associated with the medication daily profile, and the controller is adapted to automatically update the daily profile adjustments user interface depending upon the medication selected.

39. The programming device for an implantable drug pump of claim 37, wherein the daily profile adjustments user interface includes a total daily dosage field, and the controller is adapted to automatically recalculate and display an updated total daily dosage within the total daily dosage field after an adjustment to the medication daily profile is made.

40. The programming device for an implantable drug pump of claim 37, wherein the daily profile adjustments user interface allows the user to make adjustments to a base rate and/or temporary rate for a medication associated with the medication daily profile.

41. The programming device for an implantable drug pump of claim 37, wherein the controller adjusts a graphical representation of the medication daily profile in response to the adjustments.

42. The programming device for an implantable drug pump of claim 37, wherein the daily profile adjustments user interface allows the user to specify and add or remove multiple temporary rate intervals to the medication daily profile.
